(19) United States
(12) Patent Application Publication
(10) Pub. No.: US 2013/0149295 A1
(43) Pub. Date: Jun. 13, 2013

(54) FURIN AND BIOLOGICALLY ACTIVE DERIVATIVES THEREOF FOR USE IN THE PREVENTION OR TREATMENT OF AN INFLAMMATORY DISEASE

(71) Applicants: Martine Cohen-Solal, Paris (FR); Abdel-Majid Khatib, Paris (FR)

(72) Inventors: Martine Cohen-Solal, Paris (FR); Abdel-Majid Khatib, Paris (FR)

(21) Appl. No.: 13/697,390

(22) PCT Filed: May 12, 2011
(86) PCT No.: PCT/EP2011/057694

§ 371 Date: Feb. 26, 2013

(30) Foreign Application Priority Data
May 12, 2010 (EP) 10305511.7

(51) Int. Cl. C12N 9/64; A61K 31/56; A61K 38/48; A61K 45/06

(52) U.S. Cl. CPC C12N 9/6424; A61K 45/06; A61K 31/56; A61K 38/482
USPC 424/94.64

(57) ABSTRACT

The present invention relates to the prevention or therapy of inflammatory diseases. More particularly, the invention relates to an isolated polypeptide comprising the subtilisin-like catalytic domain of the furin or a biologically active derivative thereof, for use in the prevention or treatment of an inflammatory disease.

### FIELD OF THE INVENTION

The invention relates to the prevention or therapy of inflammatory diseases. More particularly, the invention relates to an isolated polypeptide comprising the subtilisin-like catalytic domain of the furin or a biologically active derivative thereof, for use in the prevention or treatment of an inflammatory disease.

### BACKGROUND OF THE INVENTION

Rheumatoid arthritis (RA) is a disease characterised by chronic inflammation of the synovial membrane. The development of the pannus in the joint causes profound and irreversible damage of cartilage and bone. The inflammation results in the influx of immune cells that secrete pro-inflammatory and anti-inflammatory cytokines (Schett et al. 2008). Therefore, the destruction of joint structures is a consequence of an imbalance between catabolic and anabolic pathways induced by cytokines. This is mediated by matrix-degrading enzymes (matrix metalloproteinases and aggrecanases) produced by effector cells of joint tissues such as fibroblasts, chondrocytes and osteoclasts. Among cells of the immune system, T lymphocytes play a major role in the inflammatory response. Key regulators are T helper (Th) a hallmark of rheumatoid arthritis. These cells secrete cytokines, soluble mediators which orchestrate the immune response. The balance is shifted to Th1 cells which produce inflammatory cytokines (IL1, TNF) while lower activation of Th2 cells promote reduced production of anti-inflammatory cytokines such as IL4. Disruption of the Th1/Th2 balance is the background of pathogenesis of human RA and in animal models of arthritis (Schulze-Koops et al. 2001). Moreover, Treg lymphocytes have been shown to play a predominant role in the pathogenesis of RA (Esensten et al. 2009). Treg are reduced in mice with RA while Treg transplantation led to a decreased severity of arthritis. So far, therapies targeted against inflammatory cytokines are available with significant protective effects on structural damage. However, full protection is not achieved yet and the persistence of mild synovitis is still responsible for joint destruction in patients receiving anti-cytokine therapies (Kraan et al. 1998), suggesting the necessity of developing an alternative approach to cytokine secreted by effector cells.

Pro-protein convertases are candidate molecules which might play a role in joint destruction in RA. PCs target several subtracts such as growth factor, integrins and metalloproteases, all being involved the pathogenesis of cancer (Scamuffa et al. 2006). The cleavage of inactive protein precursors by PC leads to active forms of MMP. Furin is an ubiquitinuous PC involved in the maturation of cartilage matrix proteins. By interacting with MMP pro-domain, furin increased the secretion of mature MMP. Its role in inflammation and in joint degradation has been demonstrated ex vivo (Milner et al. 2003). Recently, it has been shown that the inhibition of furin activates immune response and reduces peripheral tolerance (Pesu et al. 2008).

Moreover, international patent application N° WO 91/06314 describes the use of furin as a medicament and relates to a pharmaceutical composition comprising an endoproteolytically active amount of furin or a furin-like enzyme, or a fragment or derivative thereof. Accordingly, patients deficient in an endoprotease may be treated by administering furin, so that an adequate processing of precursor proteins is yet possible.

More recently, US patent application N° US 2004/0127396 relates to the use of furin and furin-like protease inhibitors, notably a α1-antitrypsin variant called PDX, in the treatment of inflammatory or matrix remodelling diseases such as rheumatoid arthritis.

### SUMMARY OF THE INVENTION

On the contrary, the inventors have now demonstrated in vivo that furin surprisingly leads to a protection mechanism in inflammatory diseases. These findings allow to propose new means of prevention or treatment of inflammatory diseases, in particular through the induction of T regulatory cell populations know as playing a role in the immune system homeostasis and tolerance to self-antigens by the administration of furin.

Thus, the invention relates in a first aspect to an isolated polypeptide comprising the subtilisin-like catalytic domain of the furin consisting of the amino acid sequence ranging from positions 108 to 464 of SEQ ID NO: 1 or a biologically active derivative thereof, for use in the prevention or treatment of an inflammatory disease.

In a second aspect, the invention relates to an activator of furin expression for use in the prevention or treatment of an inflammatory disease.

In a third aspect, the invention relates to an activator of furin activity for use in the prevention or treatment of an inflammatory disease.

In a fourth aspect, the invention relates to a pharmaceutical composition for use in the prevention or treatment of an inflammatory disease, comprising an isolated polypeptide according to the invention or a biologically active derivative thereof, an activator of furin expression or an activator of furin activity.

In a fifth aspect, the invention relates to a kit for treating an inflammatory disease comprising a first pharmaceutical composition according to invention and a second pharmaceutical composition comprising one or more therapeutically active agents selected from the group consisting of non-steroidal anti-inflammatory agents, corticosteroids, and disease modifying anti-rheumatic drugs.

### DETAILED DESCRIPTION OF THE INVENTION

### Definitions

Throughout the specification, several terms are employed and are defined in the following paragraphs.

As used herein, the term “furin” (also known as paired basic amino acid cleaving enzyme or PACE) has its general meaning in the art and refers to an enzyme which belongs to the subtilisin-like proprotein convertase (PC) family. Similar to many other proteinases, furin is synthesized as a zymogen (profurin) which becomes active only after the autocatalytic removal of its auto-inhibitory prodomain after its deposition into the appropriate cellular compartment, namely the rough endoplasmic reticulum (RER). The term may include naturally occurring furin and variants and modified forms thereof The furin can be from any source, but typically is a mammalian (e.g., human and non-human primate) furin, particularly a human furin. The naturally occurring profurin protein has an aminoacid sequence as shown in Genbank Accession number NP_{—}002560 and represented by SEQ ID NO: 1.

As used herein, the term “subtilisin-like catalytic domain of the furin” refers to the amino-terminal profurin fragment comprising the amino acid sequence ranging from positions 108 to 464 of SEQ ID NO: 1. Said subtilisin-like catalytic domain is responsible for the proteolytic activity of furin.

As used herein, the term “biologically active derivatives of a polypeptide comprising the subtilisin-like catalytic domain of the furin” includes the variants and the fragments of the polypeptide to which it refers and that retain the biological activity and the specificity of the parent polypeptide. Therefore, the “derivatives of the a polypeptide comprising the subtilisin-like catalytic domain of the furin” include variants and fragments of the polypeptide consisting of the amino acid sequence ranging from positions 108 to 464 of SEQ ID NO: 1. Thus, the “biologically active” derivatives of a polypeptide comprising the subtilisin-like catalytic domain of the furin have to show a high biological capacity to specifically cleave substrate possessing an R—X—X—R motif and more particularly an R—X—K/R—R motif as described in Takahashi et al. 1994. Preferably, the proteolytic activity of the derivatives of a polypeptide comprising the subtilisin-like catalytic domain of the furin has to be of at least about 70%, preferably between 80% and 90%, more preferably between 90% and 99%, and even more preferably 100% of the proteolytic activity of the parent polypeptide, as assessed in vitro by conventional techniques such as furin activity assays using fluorogenic substrates as previously described in Basak et al. 2009 and Scamuffa et al. 2008.

An “activator of gene expression” refers to a natural or synthetic compound that has a biological effect to enhance or significantly increase the expression of a gene. Consequently an “activator of furin gene expression” refers to a natural or synthetic compound that has a biological effect to significantly increase the expression of the gene encoding for furin. Preferably, to be considered as efficient activator, a compound must enhance the expression of the furin protein by at least 50% or enough to increase significantly the levels of furin substrates processing. The level expression of furin protein may assessed by conventional techniques such as standard electrophoretic and immunodiagnostic techniques, including immunoassays such as competition, direct reaction, or sandwich type assays (e.g. Western blots or enzyme-labeled and mediated immunoassays, such as ELISAs).

An “activator of gene activity” refers to a natural or synthetic compound that has a biological effect stimulating or enhancing the furin activation pathway. The furin activity could be evaluated by various assays such as those described in Scamuffa et al. 2008. For instance, a first assay is based on the ability of cells to mature established furin substrates such as PDGF-A or IGF-IR in cells and revealed by western blotting or biosynthetic labeling. A second assay is an in vitro enzymatic digestion assay designed to assess the level of digestion of any furin fluorogenic substrate.

### Therapeutic Methods and Uses

The present invention provides methods and compositions (such as pharmaceutical compositions) for use in the prevention or treatment of an inflammatory disease.

In a first aspect, the present invention relates to an isolated polypeptide comprising the subtilisin-like catalytic domain of the furin consisting of the amino acid sequence ranging from positions 108 to 464 of SEQ ID NO: 1 or a biologically active derivative thereof, for use in the prevention or treatment of an inflammatory disease.

In one embodiment, the isolated polypeptide of the invention consists of the subtilisin-like catalytic domain of the furin consisting of the amino acid sequence ranging from positions 108 to 464 of SEQ ID NO: 1.

In another embodiment, the isolated polypeptide of the invention consists of mutant furin lacking the transmembrane domain and the cytoplasmic tail called “shed” furin as well as other furin mutants such as mutants lacking also the cysteine rich region described in the international patent application WO 01/94383, which is incorporated therein by reference.

In still another embodiment, the isolated polypeptide of the invention consists of the mature form of furin.

In a preferred embodiment, the isolated polypeptide of the invention consists of profurin protein itself represented by SEQ ID NO: 1.

The polypeptides according to the invention may be obtained through conventional methods of solid-phase chemical polypeptide synthesis or alternatively through conventional methods based on recombinant DNA technology, e.g., through a method that, in brief, includes inserting the nucleic acid sequence coding for one polypeptide of the invention into an appropriate plasmid or vector, transforming competent cells for said plasmid or vector, and growing said cells under conditions that allow the expression of the polypeptide of the invention and, if desired, isolating and (optionally) purifying said polypeptide of the invention through conventional means known to experts in these matters. The nucleic acid sequence that codes for one polypeptide of the invention may be easily deduced from the correspondence that exists between the amino acids and the nucleotide codons that code for such amino acids. In this case, an additional aspect of the invention is an isolated nucleic acid sequence that codes for the polypeptide of the invention. In one particular embodiment, said nucleic acid is selected from single-strand DNA, double-stranded DNA, and RNA. Additional aspects of this invention are plasmids and expression vectors that contain said nucleic acid sequence that codes for one polypeptide of the invention, as well as prokaryotic or eukaryotic cells that express said polypeptide. A review of the principles of recombinant DNA technology may be found, for example, in the text book entitled “Principles of Gene Manipulation: An 5 Introduction to Genetic Engineering,” R. W. Old & S. B. Primrose, published by Blackwell Scientific Publications, 4th Edition (1989).

It must be further noted that the present invention also encompasses biologically active derivatives of the above-defined polypeptides. As used herein, the term “derivatives” includes the variants and the fragments of the polypeptide to which it refers. More particularly, the derivatives refer to “biologically active” variants and fragments of this polypeptide, i.e. variants and fragments retaining the biological activity and the specificity of the parent polypeptide comprising the subtilisin-like catalytic domain of the furin.

Accordingly, in a preferred embodiment, the derivative of the polypeptide comprising the subtilisin-like catalytic domain of the furin is a biologically active fragment thereof.

In one embodiment, the biologically active fragment is a polypeptide comprising the amino sequence ranging from positions 153 to 194 of SEQ ID NO: 1 fused to the amino sequence ranging from positions 295 to 368 of SEQ ID NO: 1 as described in Henrich et al. 2003.

In another preferred embodiment, the derivative of the polypeptide comprising the subtilisin-like catalytic domain of the furin is a biologically active variant thereof.

Said variant can be either an allelic variant of the polypeptide, or a peptidomimetic of the polypeptide. An “allelic variant of the polypeptide” has the same amino acid sequence as the polypeptide comprising the subtilisin-like catalytic domain of the furin in, except that one or more amino acids have been replace by other amino acids or suppressed, the final polypeptide retaining the biological activity and specificity of the parent polypeptide. Preferably, such allelic variant has 70%, preferably 80%, more preferably 90% and even more preferably 95% of identity as compared with the parent polypeptide.

The variant of the polypeptide may also be a peptidomimetic variant, which is an organic molecule that mimics some properties of the parent polypeptide, including at least one or more properties of interest that preferably is its biological activity. Preferred peptidomimetics are obtained by structural modification of furin, preferably using unnatural amino acids, D-amino acid instead of L amino acids, conformational restraints, isosteric replacement, cyclization, or other modifications. Other preferred modifications include, without limitation, those in which one or more amide bond is replaced by a non-amide bond, and/or one or more amino acid side chain is replaced by a different chemical moiety, or one or more of the N-terminus, the C-terminus or one or more side chain is protected by a protecting group, and/or double bonds and/or cyclization, and/or stereospecificity is introduced into the amino chain to increase rigidity and/or binding affinity. Still other preferred modifications include those intended to enhance resistance to enzymatic degradation, improvement in the bioavailability, and more generally in the pharmacokinetic properties, compared to the parent polypeptide. All of these variations are well known in the art. Thus, given the sequence of parent polypeptide, those skilled in the art are enabled to design and produce peptidomimetics having biological characteristics similar or superior to said polypeptide.

In one embodiment, the derivative of the polypeptide comprising the subtilisin-like catalytic domain of the furin is a modified furin polypeptide with improved characteristics as described in international patent application WO 01/94383 and in Plaimauer et al. 2001, which are incorporated therein by reference. Other examples of derivatives are encompassed in the context of the invention such as those described in Sucic et al. 1998, Takahashi et al. 1995, Creemers et al. 1995 and Zhou et al. 1998, which are incorporated therein by reference.

In a second aspect, the present invention also relates to an activator of furin expression for use in the prevention or treatment of an inflammatory disease.

In one embodiment, activators of furin expression are compounds that transactivate the fur gene P1 promoter. Accordingly, activators of the expression of furin include transcriptions factors for instance by C/EBPβ, GATA-1, SMADs, HIF-1 and CDX2 transcription factors.

In a third aspect, the present invention further relates to an activator of furin activity for use in the prevention or treatment of an inflammatory disease.

Preferably, the inflammatory disease is selected in the group consisting of arthritis, e.g. rheumatoid arthritis (RA), spondylarthopathies, psoriatic rheumatism, systemic lupus erythematosus (SLE) and Sjogren's syndrome.

More preferably, the inflammatory disease is rheumatoid arthritis.

The invention also relates to a method of preventing or treating an inflammatory disease comprising the step of administering an effective amount of an isolated polypeptide comprising the subtilisin-like catalytic domain of the furin consisting of the amino acid sequence ranging from positions 108 to 464 of SEQ ID NO: 1 or a biologically active derivative thereof as described herein to an subject in need thereof.

The invention further relates to a method of preventing or treating an inflammatory disease comprising the step of administering an effective amount of an activator of furin expression or an activator of furin activity as described herein to an subject in need thereof.

As used herein, the term “subject” denotes a mammal, such as a rodent, a feline, a canine, and a primate. Preferably, a subject according to the invention is a human.

Said subject in need thereof preferably suffers from or is at risk of suffering from an inflammatory disease.

### Screening Methods

Biologically active derivatives (fragments and variants) of the invention useful in the prevention or the treatment of an inflammatory disease may be identified by screening methods described in the state of the art. The screening methods of the invention can be carried out according to known in a first time by in vitro methods such as furin activity assays as described in Gawlik et al. 2009.

The biologically active derivatives that have been positively selected at the end of the in vitro screening may be subjected to further selection steps in view of further assaying its anti-inflammatory properties. By way of example, said biologically active derivatives having the biological properties of the parent polypeptide may easily be screened for instance with an in vivo murine Collagen-Induced Arthritis (CIA) model. Other models can be used such as KRN/NOD or TNF transgenic mice.

### Pharmaceutical Compositions

The polypeptides and biologically active derivatives as well as compounds described herein may be formulated into a pharmaceutical composition. Thus the invention contemplates a pharmaceutical composition comprising any one of the above polypeptides or compounds and a physiologically acceptable carrier. Physiologically acceptable carriers can be prepared by any method known by those skilled in the art.

Pharmaceutical compositions comprising at least one polypeptide or compound of the invention include all compositions wherein the polypeptide(s) or compound(s) are contained in an amount effective to achieve the intended purpose. In addition, the pharmaceutical compositions may contain suitable pharmaceutically acceptable carriers comprising excipients and auxiliaries which facilitate processing of the active compounds into preparations which can be used pharmaceutically. Suitable pharmaceutically acceptable vehicles are well known in the art and are described for example in Remington's Pharmaceutical Sciences (Mack Publishing Company, Easton, USA, 1985), which is a standard reference text in this field. Pharmaceutically acceptable vehicles can be routinely selected in accordance with the mode of administration, solubility and stability of the polypeptides or compounds. For example, formulations for intravenous administration may include sterile aqueous solutions which may also contain buffers, diluents and other suitable additives. The use of biomaterials and other polymers for drug delivery, as well the different techniques and models to validate a specific mode of administration, are disclosed in literature.

The polypeptides or compounds of the present invention may be administered by any means that achieve the intended purpose. For example, administration may be achieved by a number of different routes including, but not limited to subcutaneous, intravenous, intradermal, intramuscular, intraperitoneal, intracerebral, intrathecal, intranasal, oral, rectal, transdermal, buccal, topical, local, inhalant or subcutaneous use.

Dosages to be administered depend on individual needs, on the desired effect and the chosen route of administration. It is understood that the dosage administered will be dependent upon the age, sex, health, and weight of the recipient, concurrent treatment, if any, frequency of treatment, and the nature of the effect desired. The total dose required for each treatment may be administered by multiple doses or in a single dose.

Depending on the intended route of delivery, the polypeptides or compounds may be formulated as liquid (e.g., solutions, suspensions), solid (e.g., pills, tablets, suppositories) or semisolid (e.g., creams, gels) forms.

In a preferred embodiment, the compositions are presented in unit dosage forms to facilitate accurate dosing. The term “unit dosage forms” refers to physically discrete units suitable as unitary dosages for human subjects and other mammals, each unit containing a pre-determined quantity of active material calculated to produce the desired therapeutic effect, in association with a suitable pharmaceutical excipient. Typical unit dosage forms include pre-filled, pre-measured ampoules or syringes of the liquid compositions or pills, tablets, capsules or the like in the case of solid compositions. In such compositions, the compound of the invention is usually a minor component (from about 0.1 to about 50% by weight or preferably from about 1 to about 40% by weight) with the remainder being various vehicles or carriers and processing aids helpful for forming the desired dosing form.

The polypeptides or compounds of this invention can also be administered in sustained release forms or from sustained release drug delivery systems.

The expression “physiologically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution. Besides the pharmaceutically acceptable carrier, the compositions of the invention may also comprise minor amounts of additives, such as stabilizers, excipients, buffers and preservatives.

The invention also contemplates a pharmaceutical composition comprising a nucleic acid encoding the polypeptide of the invention in the frame of e.g. a treatment by gene therapy. In this case, the nucleic acid is preferably present on a vector, on which the sequence coding for the peptide is placed under the control of expression signals (e.g. a promoter, a terminator and/or an enhancer) allowing its expression. The vector may for example correspond to a viral vector such as an adenoviral or a lentiviral vector.

The polypeptides and biologically actives derivative thereof, the activators of furin expression or the activators of furin activity may also be used in combination with other therapeutically active agents, for instance, non-steroidal anti-inflammatory agents, corticosteroids, and disease modifying anti-rheumatic drugs.

Such disease modifying anti-rheumatic drugs include but are not limited to tumor necrosis factor (TNF) inhibitors such as etanercept (Enbrel®), infliximab (Remicade®) and adalimumab (Humira®), IL-1 receptor antagonists (IL1ra) such as anakinra (Kineret®), B cell depleting agents such as Rituximab (Rituxan®), anti-IL-6 antibodies (Tocilizumab, Roactemra ®), T-cell costimulatory blockers such as abatacept (Orencia®) as well as other drugs such as for instance methotrexate, sulfasalazine, leflunomide, antimalarials, gold salts, d-penicillamine, cyclosporin A, cyclophosphamide and azathioprine.

The foregoing therapeutically active agents are listed by way of example and are not meant to be limiting. Other therapeutically active agents which are currently available or that may be developed in the future are equally applicable.

Accordingly, pharmaceutical compositions of the invention as described above may further comprise one or more therapeutically active agents selected in the group consisting of non-steroidal anti-inflammatory agents, corticosteroids, and disease modifying anti-rheumatic drugs. In one preferred embodiment, said therapeutically active agent is selected in the group consisting of tumor necrosis factor (TNF) inhibitors, IL-1 receptor antagonists (IL1ra), B cell depleting agents and T-cell costimulatory blockers.

Moreover, if they are contained in different pharmaceutical compositions, said compositions may be administered to the patient at the same time or successively. Thus, the present invention also relates to a kit for the prevention or treatment of an inflammatory disease comprising a first pharmaceutical composition comprising a polypeptide or a compound according to the invention and a second pharmaceutical composition comprising one or more therapeutically active agents selected from the group consisting of non-steroidal anti-inflammatory agents, corticosteroids, and disease modifying anti-rheumatic drugs.

The invention will be further illustrated by the following figures and examples. However, these examples and figures should not be interpreted in any way as limiting the scope of the present invention.

### FIGURES

FIG. 1: Evolution of clinical arthritis score: After induction of arthritis, mice received intra-peritoneous injections of furin, or α1-PDX or furin twice a week until sacrifice. Furin decreased significantly the arthritis score while α1-PDX worsened this score.

FIG. 2: Histological score and correlation with clinical score:

A: At sacrifice, furin decreased significantly histological score whereas α1-PDX induced a significantly higher score.

B: There was a positive correlation between clinical arthritis score and histological score at sacrifice.

FIG. 3: Percentage of Treg in the spleen in different conditions:

The percentage of Treg was measured in spleen mice at sacrifice. The percentage decreased in mice with CIA treated with PBS. Furin induced an increased in percentage of Treg, while α1-PDX reduced this percentage.

FIG. 4: Changes in BMD in the 4 groups of mice:

A: Bone mineral density (BMD) was measured at the whole body at baseline and sacrifice. The change in BMD was lower in the group of CIA mice treated with PBS. Furin was able to reverse the bone loss whereas α1-PDX worsened the bone loss.

B: A significant negative correlation was found between the change of BMD and the clinical arthritis score (p<0.01).

### MATERIAL & METHODS

Induction of CIA in mice and treatment: Male mice of DBA/1 strain, which is susceptible to CIA, were purchased from Janvier (France) at 5-7 weeks of age. In vivo experiments complied with the recommendations for animal experimentation issued by the National Institutes for Health and by our local Ethics Committee on Animal Care and Experimentation. After one week housing, the mice were immunized as described previously (Saidenberg-Kermanac'h et al. 2004). Briefly, each mouse received a subcutaneous injection at the base of the tail of 100 μg of native bovine collagen type II (Chondrex, Morwell Diagnostic, Zurich, Switzerland) emulsified in complete Freund's adjuvant (CFA). A subcutaneous booster of 100 μg of collagen type II in incomplete Freund's adjuvant was given 21 days later.

There were then separated into 3 groups of 7-10 mice. The groups with CIA were given one of the following treatments 3 times a week IP from arthritis onset to sacrifice:
CIA mice treated with saline solution (PBS group)
CIA mice treated with Furin at the dose of 1 U (Furin group)
CIA mice treated with α1-PDX at the dose of 14 μg (α1-PDX group)
A group of mice was not immunized and was used as naïve.
Experiments were performed 4 times with a mean number of 3-7 mice per group.
Furin was purchased at New England Biolabs, France (ref P8077L) and α1-PDX (RP-070) at Ozyme (France).

Assessment of arthritis: From day 31 to sacrifice, collagen induced arthritis paws were scored 3 times a week using a clinical score in a blind manner. Clinical severity of arthritis in each joint or group of joints (toes, tarsus, and ankles) was scored as follows: 0: normal; 1: erythema; 2, swelling: 3: deformity and 4: necrosis. These scores were summed to obtain the arthritis score; the mean arthritis score in each group was used to evaluate CIA severity on each clinical observation day. The animals were sacrificed 36 days after immunization when arthritis reached a plateau.

Histological score: At sacrifice, the legs were dissected free and processed for histological studies. One paw was collected into a solution of PFA 4%, and then decalcified in PFA 1%-EDTA 0.2M for 3 weeks renewing the solution 3 times a week. Samples were embedded in paraffin and at least four serial sections were cut from each paw to ensure extensive evaluation of the arthritic joints. Evaluation was done in a blind manner in each slice using 4 parameters: synovial inflammation, synovial thickness and invasion into the joint, cartilage unevenness, bone erosion according to a four-point scale (0-3, where 0 indicated a normal joint and 3 maximally severe arthritis).

Cytokine measurements: One paw of each mouse was collected at sacrifice in order to measure the cytokine secretion in the whole joint. After dissection of soft tissues, whole joint was grounded in a protein buffer and assayed for cytokines. Pro-inflammatory cytokines (TNF-α, IL-1) and anti-inflammatory cytokines (IL-4, IL-10) were measured in the whole paw and in the supernatant of splenocyte cultures using ELISA assays (R&D system). Levels were expressed in μg/mg of protein.

Flow cytometric analysis: Treg lymphocyte profile was assessed in the spleen cells of 3 mice from each treatment group and in the naïve mice. Forteen days after the first boost, the spleens were collected and cells were isolated for FACS analysis. Spleens were collected on ice and prepared for cell suspensions into complete medium containing RPMI and 20% serum (Gibco, France) supplemented with 20 U/ml penicillin, 0.02 mg/ml streptomycin and 2 mM L-glutamine (Sigma, France) by mechanical mashing through a 40 μm cell strainer (Falcon). Red blood cells were lysed using Gey buffer, then washed out with RPMI with 20% serum.

Splenocytes were labeled with PerCP-Cy5.5-conjugated anti-CD4 and FITC-conjugated anti-CD25. An intracellular FoxP3 staining was performed with PE-conjugated anti-mouse/rat FoxP3 according to BD bioscience protocol. Cells were analysed using a FACSCalibur (BD Pharmingen) and data were analysed with Cellquest software. Data are expressed as the percentage of gated events.

Evaluation of bone parameters: To assess the effects of Furin and α1-PDX on systemic bone loss, we measured whole-body bone mineral density (BMD, g/cm2) at baseline (before immunization) and at sacrifice. BMD was measured using a Lunar Piximus device and the BMD change from baseline to sacrifice (ABMD) were calculated as (BMD at sacrifice—BMD at baseline)/BMD at baseline and expressed in percentage.

MMP array: Protein antibody arrays were performed to assess the expression of MMP in mouse paws (Protein antibody arrays Biotin Label-based Mouse Antibody Array I ref: AAM-BLM-1-4, Raybiotech) according to the manufacturer's instructions. Briefly, 50 μg of protein from each paw extract were biotin-labelled and loaded on antibody-coated membrane. Then horseradish peroxidase-conjugated streptavidin based detection is performed with FujiFilm LAS-3000 Intelligent dark box (Fuji, France). Measurement of integrated optical density dot is assessed with ImageJ after retrieval of non-specific optical signal density. Each measurement is a ratio between mean of dot of interest and the mean of six membrane-internal control dots.

Zymography: Gelatinase activity of MMP-2 and MMP-9 were assayed in culture medium and total paws using gelatin zymography. From each sample, 6 μg of protein normalized with BCA method was separated by SDS/PAGE in a 10% (w/v) gel containing 1 mg/ml gelatin. The gel was washed for 30 minutes in a 2.5% (w/v) triton 100×, 40 mM Tris-HCl pH 7.8 buffer at room temperature. Zymography gel is then incubated over night at 37° C. in activation buffer containing 50 mM Tris-HCl pH 8, 10 mM CaC12. Finally staining in Coomassie Blue 0.5% was performed for 1 hour followed by 30 min destaining in 20% (w/v) ethanol, 10% (w/v) glacial acetic acid. Pictures of the gels were taken with FujiFilm LAS-3000 Intelligent dark box (Fuji, France).

Statistical analysis: Results were expressed as means±S.E.M. Comparison tests were performed using analysis of variance for repeated measurements (ANOVA) followed by Fisher PLSD tests when appropriate.

Results

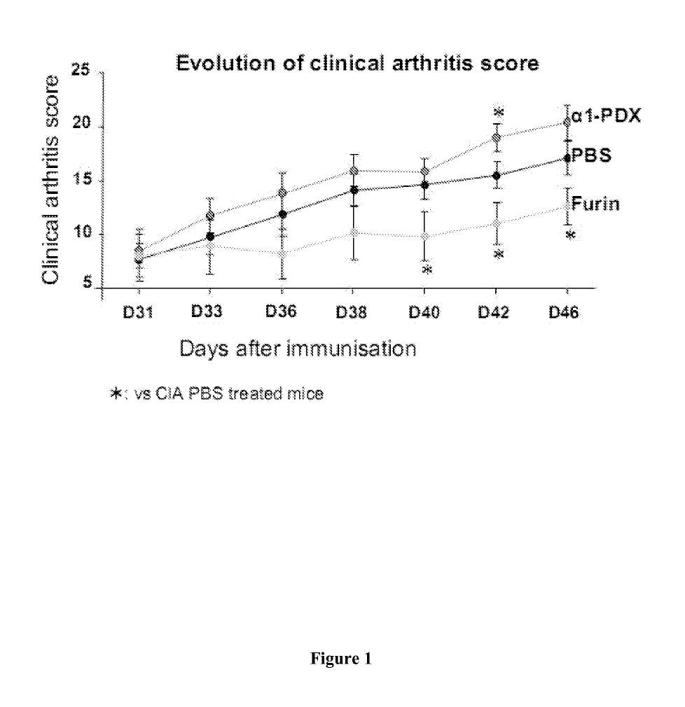

Effects of Furin and its inhibition in the development of arthritis: Ten days after the second immunization boost, arthritis begun in all group. Furin decreased significantly the arthritis score from day 40 compared to PBS (9.7±3.26 vs 14.75±1.45, p<0.05). From day 42, arthritis score was significantly lower in mice treated with Furin, while it was higher in those which received α1PDX both compared to PBS-treated mice (10.68±2.74; 20.31±1.11 vs 15.63±1.78 respectively, p<0.05). At time of sacrifice, arthritis score was still significantly reduced in mice which received Furin (12.07±2.38) whereas it was higher in those which received α1-PDX (20.49±1.77) compared to PBS-treated mice (16.98±1.63, p<0.05) (FIG. 1).

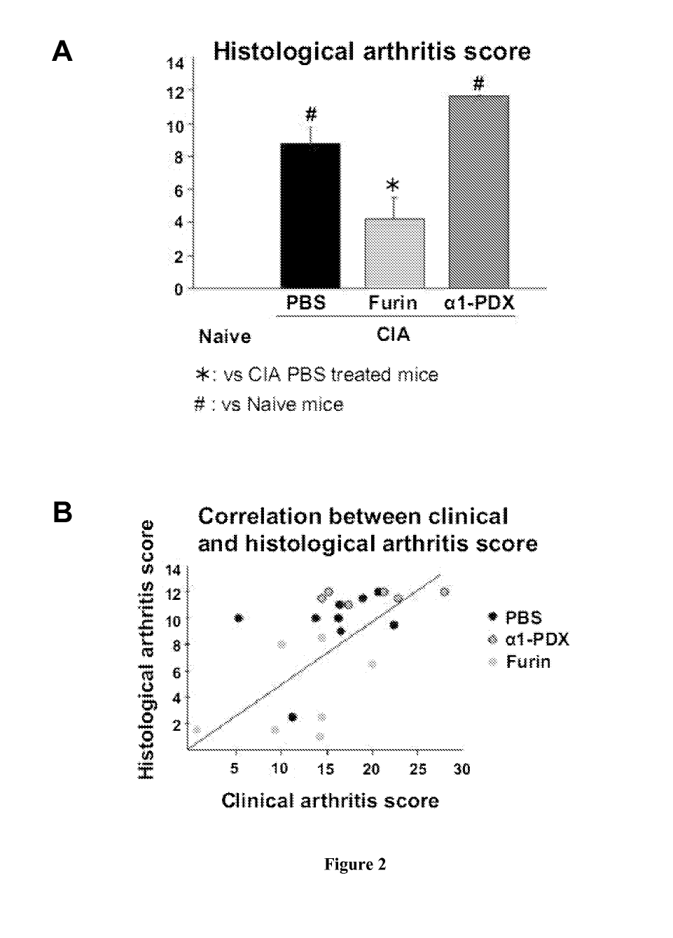

Histological score in the presence of Furin or its inhibition: Histological scoring was performed at sacrifice. All immunized mice displayed histological lesions. Compared to PBS-treated mice, Furin 1 U decreased histological score while α1-PDX worsened the score (8.75±1.121 vs 4.21±1.253 and 11.67±0.16, p<0.01 and <0.05 respectively, FIG. 2A). Moreover, histological score was correlated to arthritis score (r=0.70, p<0.01, FIG. 2B).

Effect of Furin in cytokine production in the joint: Cytokines were measured in the whole paw to assess the local secretion. In mice treated with α1-PDX, the levels of pro-inflammatory cytokines IL1β and TNFα levels were significantly higher compared to PBS group (15.567±4.485 vs 4.4±0.252 and 28.933±2.815 vs 16.367±2.373). In contrast, the levels of anti-inflammatory cytokines IL10 and IL4 were decreased (7.267±0.498 and 6.9±0.361 vs 15.933±0.203 pg/mg). Results are summarized in Table 1:

TABLE I
Cytokine content in the mice paw from each group

| | PBS | Furin | α1-PDX |
|---|---|---|---|
| TNFα | 14.880 ± 0.908 | 6.880 ± 1.184**† | 22.503 ± 3.466** |
| (pg/mg prot) | | | |
| IL1β | 5.322 ± 0.360 | 2.54 ± 0.309† | 12.733 ± 2.831*** |
| (pg/mg prot) | | | |
| IL4 (pg/mg | 14.397 ± 0.445 | 24.040 ± 2.485***† | 9.767 ± 2.668* |
| prot) | | | |
| IL10 (pg/mg | 15.194 ± 0.330 | 21.320 ± 1.179***† | 9.033 ± 1.972*** |
| prot) | | | |

*vs PBS treated-mice
*P < 0.05;
**p < 0.01; p < 0.001
† P < 0.0001 vs a1PDX treated group.

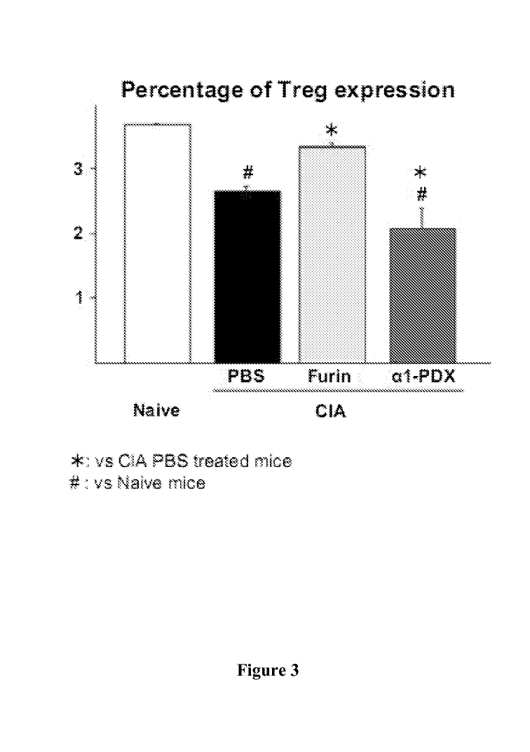

Effect of Furin in Treg expression: The induction of arthritis results in a diminution of the number of Treg compared to naïve mice (2.71±0.07 vs 3.74±0.02%, p<0.005). Treg number was also affected by Furin and α1-PDX (FIG. 3). Compared to CIA PBS-treated mice, Furin 1 U increased the number of Treg (2.71±0.07 vs 3.39±0.05%, p=0.04) while α1-PDX decreased the percentage of Treg (2.71±0.07 vs 2.11±0.32%, p=0.05). Moreover, the expression of Treg reached the level of naïve mice.

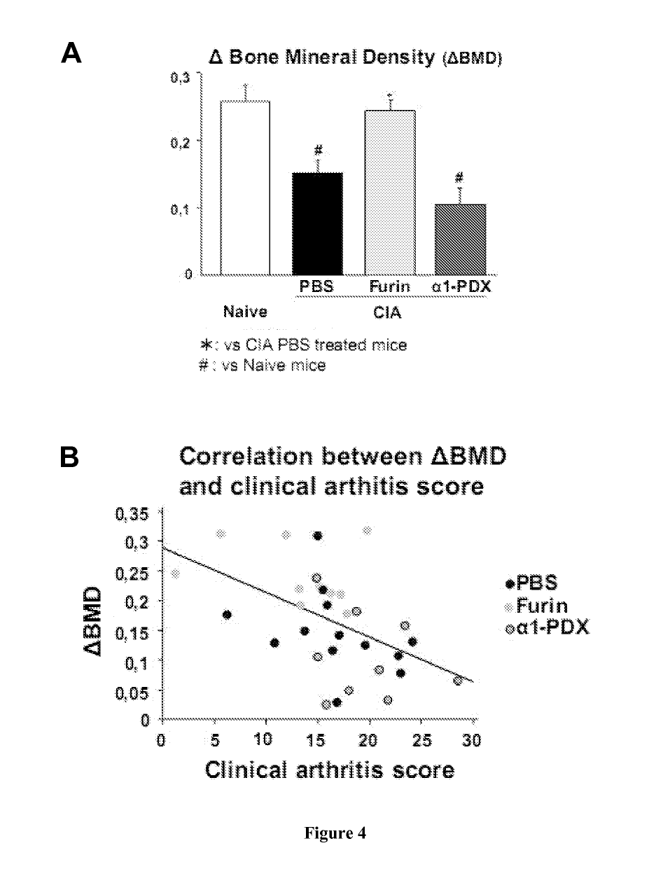

Effects of Furin in inflammatory-related bone loss: As expected, bone loss occurs in CIA mice treated with PBS. Bone mineral density is significantly higher in Furin group than in PBS (0.243±0.017 vs 0.152±0.018 g/cm2, p<0.01) until reaching the level of naïve group. BMD was lower in α1-PDX group than in PBS-group (0.105±0.025 vs 0.152±0.018 g/cm2, p=0.15). Furthermore the bone mineral density is correlated with the arthritis score (FIGS. 4A and 4B).

Effects of Furin in expression and activity of MMPs: Effect of systemic administration of Furin and α1-PDX in the expression and activity of MMPs reported to be involved in joint destruction was also investigated. MMP-2, MMP-9 and MMP-14 expression were up-regulated in inflammatory conditions while Furin and α1-PDX decreased and enhanced their expression respectively. Accordingly, immuno-histochemistry showed a high expression of the MMPs in the pannus of arthritic mice. Moreover, Furin results in a decreased MMP expression whereas enhanced with α1-PDX and this was related to the thickness of the synovial pannus. We then assessed the catalytic functions of MMP by zymography. The analysis of gelatinolytic activity in the joint revealed that the majority of MMP-2 and MMP-9 were found under precursor forms in naïve mice and that arthritis was associated with the accumulation of the processed active forms of these MMPs, indicating the maturation process. The increased processing of MMP-2 and MMP-9 was previously linked to Furin activity (Lalou et al. 2010). Here, Furin inhibited the accumulation of precursor forms of these MMPs and their processing. In contrast, administration of α1-PDX enhanced the processing of MMP-2. Altogether, these results indicate that the reduction of clinical and histological score observed with systemic administration of Furin is mainly due to the reduction of the synovial pannus which secreted MMP locally and suggests a systemic regulation of the formation of pannus.

### REFERENCES

Throughout this application, various references describe the state of the art to which this invention pertains. The disclosures of these references are hereby incorporated by reference into the present disclosure.

Basak A, Khatib A M, Mohottalage D, Basak S, Kolajova M, Bag S S, Basak A; A novel enediynyl peptide inhibitor of furin that blocks processing of proPDGF-A, B and proVEGF-C; PLoS One. 2009 Nov. 26; 4(11):e7700.

Creemers J W, Vey M, Schafer W, Ayoubi T A, Roebroek A J, Klenk H D, Garten W, Van de Ven W J; Endoproteolytic cleavage of its propeptide is a prerequisite for efficient transport of furin out of the endoplasmic reticulum; J Biol Chem. 1995 Feb. 10; 270(6):2695-702

Esensten J H, Wofsy D, Bluestone J A. Regulatory T cells as therapeutic targets in rheumatoid arthritis. Nat Rev Rheumatol 2009; 5(10):560-5.

Gawlik K, Shiryaev S A, Zhu W, Motamedchaboki K, Desjardins R, Day R, Remade A G, Stec B, Strongin A Y; Autocatalytic activation of the furin zymogen requires removal of the emerging enzyme's N-terminus from the active site; PLoS One. 2009; 4(4):e5031.

Gendron F P, Mongrain S, Laprise P, McMahon S, Dubois C M, Blais M, Asselin C, Rivard N. The CDX2 transcription factor regulates furin expression during intestinal epithelial cell differentiation. Am J Physiol Gastrointest Liver Physiol. 2006 February; 290(2):G310-8.

Henrich S, Cameron A, Bourenkov G P, Kiefersauer R, Huber R, Lindberg I, Bode W, Than M E; The crystal structure of the proprotein processing proteinase furin explains its stringent specificity; Nat Struct Biol. 2003 July; 10(7):520-6.

Kraan M C, Versendaal H, Jonker M, Bresnihan B, Post W J, t Hart B A, et al. Asymptomatic synovitis precedes clinically manifest arthritis. Arthritis Rheum 1998; 41(8):1481-8.

Lalou, C., et al. Inhibition of the proprotein convertases represses the invasiveness of human primary melanoma cells with altered p53, CDKN2A and N-Ras genes. PLoS One. 9, 9992 (2010).

Milner J M, Rowan A D, Elliott S F, Cawston T E. Inhibition of furin-like enzymes blocks interleukin-lalpha/oncostatin M-stimulated cartilage degradation. Arthritis Rheum 2003; 48(4): 1057-66.

Pesu M, Watford W T, Wei L, Xu L, Fuss I, Strober W, et al. T-cell-expressed proprotein convertase furin is essential for maintenance of peripheral immune tolerance. Nature 2008; 455(7210):246-50.

Plaimauer B, Mohr G, Wernhart W, Himmelspach M, Dorner F, Schlokat U; ‘Shed’ furin: mapping of the cleavage determinants and identification of its C-terminus; Biochem J. 2001 Mar. 15; 354(Pt 3):689-95.

Saidenberg-Kermanac'h N, Corrado A, Lemeiter D, deVernejoul M C, Boissier M C, Cohen-Solal M E. TNF-alpha antibodies and osteoprotegerin decrease systemic bone loss associated with inflammation through distinct mechanisms in collagen-induced arthritis. Bone 2004; 35(5):1200-7.

Scamuffa N, Calvo F, Chretien M, Seidah N G, Khatib A M. Proprotein convertases: lessons from knockouts. FASEB J 2006; 20(12):1954-63.

Scamuffa N, Siegfried G, Bontemps Y, Ma L, Basak A, Cherel G, Calvo F, Seidah N G, Khatib A M; Selective inhibition of proprotein convertases represses the metastatic potential of human colorectal tumor cells; J Clin Invest. 2008 January; 118(1):352-63.

Schett G, Stach C, Zwerina J, Voll R, Manger B. How antirheumatic drugs protect joints from damage in rheumatoid arthritis. Arthritis Rheum 2008; 58(10):2936-48.

Schulze-Koops H, Kalden J R. The balance of Th1/Th2 cytokines in rheumatoid arthritis. Best Pract Res Clin Rheumatol 2001; 15(5):677-91.

Sucic J F, Spence M J, Moehring T J; Structural and functional analysis of the protein products derived from mutant fur alleles in an endoprotease-deficient Chinese hamster ovary cell strain; Somat Cell Mol Genet. 1998 March; 24(2):75-90.

Takahashi S, Hatsuzawa K, Watanabe T, Murakami K, Nakayama K. Sequence requirements for endoproteolytic processing of precursor proteins by furin: transfection and in vitro experiments. J Biochem. 1994 July; 116(1):47-52.

Takahashi S, Nakagawa T, Kasai K, Banno T, Duguay S J, Van de Ven W J, Murakami K, Nakayama K; A second mutant allele of furin in the processing-incompetent cell line, LoVo. Evidence for involvement of the homo B domain in autocatalytic activation; J Biol Chem. 1995 Nov. 3; 270(44):26565-9.

Zhou A, Martin S, Lipkind G, LaMendola J, Steiner D F; Regulatory roles of the P domain of the subtilisin-like prohormone convertases; J Biol Chem. 1998 May 1; 273(18):11107-14.

1-7. (canceled)

8. A method of preventing or treating an inflammatory disease in a subject in need thereof, comprising
administering to said subject an effective amount of a polypeptide comprising a subtilisin-like catalytic domain of furin, wherein the subtilisin-like catalytic domain comprises an amino acid sequence ranging from positions 108 to 464 of SEQ ID NO: 1, or a biologically active derivative thereof.

9. The method of claim 8, wherein said polypeptide is a profurin represented by SEQ ID NO: 1.

10. The method of claim 8, wherein said polypeptide is a mature form of furin.

11. The method of claim 8, wherein said inflammatory disease is rheumatoid arthritis.

12. The method of claim 8, wherein said administering step provides said polypeptide to said subject together with a physiologically acceptable carrier.

13. A kit for the prevention or treatment of an inflammatory disease comprising
a first pharmaceutical composition comprising a polypeptide comprising a subtilisin-like catalytic domain of furin, wherein the subtilisin-like catalytic domain comprises an amino acid sequence ranging from positions 108 to 464 of SEQ ID NO: 1, or a biologically active derivative thereof,
and
a second pharmaceutical composition comprising one or more therapeutically active agents selected from the group consisting of non-steroidal anti-inflammatory agents, corticosteroids, and disease modifying anti-rheumatic drugs.


SEQUENCE LISTING (1 sequence)

SEQ ID NO: 1 - protein, 794 aa, Homo sapiens
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL